(19) United States
(12) Patent Application Publication
(10) Pub. No.: US 2004/0171551 A1
(43) Pub. Date: Sep. 2, 2004

(54) Molecules binding to Glu-Pro motifs, therapeutic compositions containing them and their applications

(75) Inventor: Frederic Triebel, Versailles (FR)

Correspondence Address: IP DEPARTMENT OF PIPER RUDNICK LLP, ONE LIBERTY PLACE, SUITE 4900, 1650 MARKET ST, PHILADELPHIA, PA, 19103, US

(73) Assignees: Institut Gustave Roussy - IGR, a corporation of France, Villejuif Cedex (FR); Universite Paris-Sud, a corporation of France, Orsay (FR)

(21) Appl. No.: 10/803,366

(22) Filed: Mar. 18, 2004

Related U.S. Application Data
(63) Continuation of application No. PCT/IB02/04240, filed on Sep. 17, 2002.

(30) Foreign Application Priority Data
Sep. 19, 2001 (EP) 01402406.1

(51) Int. Cl. A61K 38/16; A61K 38/10; C07K 14/47

(52) U.S. Cl. 514/12; 514/13; 530/324; 530/326

(57) ABSTRACT

A molecule binding to a target including an EP motif having the following sequence: (X-(EP)ₙ-Y-(EP)ₘ-Z)ₚ wherein X, Y and Z may be identical or different and include a sequence of 0 to 10 amino acids, identical or different, n and m are integers between 0 to 20, preferably between 3 to 10, with at least one of n or m being different from 0, and p is an integer between 1 and 10.

### RELATED APPLICATION

This is a continuation of International Application No. PCT/IB02/04240, with an international filing date of Sep. 17, 2002 (WO 03/035682, published May 1, 2003), which is based on European Patent Application No. 01401406.1, filed Sep. 19, 2001.

### FIELD OF THE INVENTION

This invention relates to molecules binding to specific targets comprising Glu-Pro (EP) repeated motifs such, for example, the lymphocyte activation gene-3 (lag-3)-associated protein hereafter named LAP. The invention also relates to therapeutic compositions containing the molecules, antibodies directed against the molecules, to therapeutical compositions containing them. Also, the invention relates to methods for screening drugs useful for the treatment of immune disorders.

### SUMMARY OF THE INVENTION

This invention relates to a molecule binding to a target including an EP motif having the following sequence: (X-(EP)ₙ-Y-(EP)ₘ-Z)ₚ wherein X, Y and Z may be identical or different and include a sequence of 0 to 10 amino acids, identical or different, n and m are integers between 0 to 20, prefereably between 3 to 10, with at least one of n or m being different from 0, and p is an integer between 1 and 10.

This invention also relates to an expression vector including a nucleic acid molecule.

This invention further relates to a method of treating immune-related pathologies including administering a therapeutically effective amount of a molecule to a patient in need thereof.

This invention still further relates to a method for screening drugs including contacting a candidate drug with a molecule in the presence of a target EP motif and measuring resulting binding of the molecule to the target.

This invention yet further relates to antibodies directed to a specific epitope of a polypeptide selected from the group consisting of polypeptides or peptides identified by SEQ ID No:1, SEQ ID NO:2, SEQ ID NO:3, SEQ ID NO:4, SEQ ID NO:5, SEQ ID NO:6 and SEQ ID NO:9.

This invention also further relates to a monoclonal antibody or a monoclonal antibody derivative that specifically binds a peptide selected from the group consisting of polypeptides or peptides identified by SEQ ID NO:1, SEQ ID NO:2, SEQ ID NO:3, SEQ ID NO:4, SEQ ID NO:5, SEQ ID NO:6 and SEQ ID NO:9, the monoclonal antibody derivative being selected from the group consisting of a monoclonal antibody conjugated to a cytotoxic agent or a radioisotope, and Fab, Fab′ or F(ab′)₂ fragments of the monoclonal antibody conjugated to a cytotoxic agent or radioisotope.

### BRIEF DESCRIPTION OF THE DRAWINGS

The invention will be described in connection with experimental results and the Figures below wherein:

FIG. 1 represents the in vitro interaction of human LAP with hLAG-3;

FIG. 1A shows that LAP binds specifically to the natural hLAG-3 (70 kDa) protein present in whole cell lysate of PHA-activated human PBMCs;

FIG. 1B shows that LAP binds specifically to a protein produced by in vitro translation of an hLAG-3 mRNA in a rabbit reticulocyte lysate;

FIG. 2 illustrates interactions tested in the two-hybrid system using co-transformation with two plasmids and mating of two yeast strains;

FIG. 2A shows three partial LAP proteins (D1, D2 and D3) lacking their C-terminal domain were cloned in frame with the GAL4 AD protein, using a partial 1104 bp LAP cDNA;

FIG. 2B shows that the EP-rich C-terminal region of the PDGF receptor (PDGFR) was fused with the LexA BD;

FIG. 2C shows interactions in the two-hybrid system; and

FIG. 3 represents Western blots autoradiograme obtained with the anti-LAP immune serum, revealing a specific band at 45 kDa. Western blots were performed using 10 μl total cell lysates of PBMC (lanes 2, 4, 6) or PHA blasts (lanes 1, 3, 5). The blots were incubated in rabbit preimmune serum (lanes 1, 2), rabbit polyclonal antibody against LAP (lanes 3, 4) or the latter preincubated with 10-6 M LAP peptide (lanes 5, 6). The arrow indicates the LAP 45 kDa protein.

### DETAILED DESCRIPTION

I previously demonstrated that both LAG-3 and MHC class II were present in the cell fraction of glycosphingolipid-rich complexes (GSL complexes) before the assembly of the immunological synapse by CD3/TCR complex crosslinking.

Using the LAG-3 intracytoplasmic region as bait in the yeast two-hybrid cloning system, I have now discovered a novel interaction between a new human protein termed LAP for LAG-3-Associated Protein and EP repeated motifs present in LAG-3. In particular, I discovered that LAP binds specifically in vitro and in vivo to the Glu-Pro (EP) repeated motif present in the LAG-3 intracytoplasmic region and that LAP also binds to the EP motif of another functionally important receptor, the PDGFR.

Such an interaction plays an important role in T cell function and homeostasis because LAG-3 acts as a negative regulator of activated T-cells and plays an important role in regulating the expansion of activated T-cells and limiting antigen induced cell death. LAG-3 associates with the TCR:CD3 complex and interferes with TCR signalling. This down regulation may be activated by disrupting CD4 and CD8 co-receptor function since LAG-3 is expressed on both CD4′ and CD8′ cells and has been shown to be associated with CD4 and CD8 in raft microdomains.

The LAP protein transduces appropriate signals that lead to this control on T cell function and CD4 and CD8 T cell subpopulation homeostasis. This negative control on T cell activation is of prime importance for regulating primary activated T-cells as well as regulating T-cell memory development and homeostasis.

LAP protein is encoded by a 1.8 kb RNA message in lymphocytes that is derived from a rare mRNA and encodes a 45 kDa protein that is expressed in most tissues. Thus, molecules that, as LAP, bind to the EP motif are candidate molecules for a new type of signal transduction and/or coupling of clustered rafts to the microtubule networks that can explain how negative signalling of co-receptors may occur through molecules devoid of immunoreceptor tyrosine-based inhibitory motifs (ITIM) consensus sequence.

Supramolecular assemblies between LAG-3, CD3, CD8 and MHC class II molecules result from the organization within raft microdomains (Hannier, S. and Triebel, F., The MHC class II ligand LAG-3 is co-distributed with CD8 and CD3/TCR molecules after their engagement by mAbs or peptide/MHC class I complexes, Int. Immunol. 1999. 11: 1745-1752). To investigate the pathway involved in LAG-3-dependent TCR signalling regulation, I directly cloned proteins expressed in activated T cells that specifically bind to the IC region of hLAG-3. Using the yeast two-hybrid system and the LAG-3 IC region as bait, I identified a novel protein, termed LAP for LAG-3-associated protein that binds to the Glu-Pro (EP) repeated motifs present within the LAG-3 IC region C-terminus.

These Glu-Pro (EP) repeated motif are present, for example, in the LAG-3 intracytoplasmic region and in the functionally important receptor named Platelet Derived Growth Factor Receptor (PDGFR). Other intracellular signalling molecules, including this unusual EP motif, are SPY75 and lckBP1 and the mouse homologues of the human HSl product. These molecules have been shown to be involved in TCR signalling.

Thus, the invention relates to molecules binding to a target comprising an EP motif, in particular, to molecules binding to a target comprising an EP motif having the following sequence:

(X-(EP)ₙ-Y-(EP)ₘ-Z)ₚ

wherein X, Y and Z may be identical or different and comprise a sequence of 0 to 10 amino acids, identical or different, n and m are integers between 0 to 20, preferably between 3 to 10, with at least one of n or m being different from 0, and p is an integer between 1 and 10.

In a preferred embodiment, the invention relates to a molecule which binds to an EP motif selected from the group comprising the following formula: EPEPEPEPEPEPEPEPEP (SEQ ID N^{o} 3), EPEPEPQLEPEP (SEQ ID N^{o} 4), EPQDEPPEPQLELQVEPEPELEQ (SEQ ID N^{o} 5), or EPEPEPEPEPEP (SEQ ID N^{o} 6).

In another preferred embodiment, the invention relates to a molecule that binds to an amino acid sequence comprising at least 5 EP motifs over a 19 amino acid length segment.

The molecule of the invention is selected from a peptide, a polypeptide or a protein. Preferably, the molecule is a purified polypeptide consisting of or comprising the amino acids sequence identified by SEQ ID No.:1, an homologous, a fragment or a derivative thereof. More preferably, the molecule is a purified polypeptide consisting of or comprising the carboxy-terminal amino acids sequence of LAP identified by SEQ ID No.:2, an homologous, a fragment or a derivative thereof.

For the purpose of the invention:

an homologous polypeptide relates to a polypeptide or a protein which can differ by one or a few amino acid residues when compared with the polypeptide of the invention, as the polypeptides identified by SEQ ID No.:1 or SEQ ID No.:2, but that maintain substantially all of the biological functions of the polypeptide, namely, its capacity to bind glu-pro motifs;

a polypeptide fragment relates to any amino acid sequence contained in the sequence of the polypeptide of the invention, which maintains the binding capacity for at Glu-Pro motifs; and

a polypeptide derivative relates to the entire or fragment polypeptides, labelled with chemical or biological entities to be easily detected. Chemical or biological entities may be enzymes, fluorescent labels, coloured particles and the like.

The invention also relates to a nucleic acid molecule consisting of or comprising a polynucleotide sequence coding a polypeptide according to the invention and, particularly, to a nucleic acid molecule coding for the polypeptide identified by SEQ ID No.;1. Also, the invention relates to a nucleic acid molecule, consisting of or comprising the polynucleotide sequence identified by SEQ ID No.:8, a fragment or a derivative thereof.

The invention relates also to an expression vector comprising a nucleic acid molecule according to invention. For the purpose of the invention, an “expression vector” refers to any replicable DNA construct used either to amplify or express DNA, which encodes one of the polypeptides of the invention.

The invention also relates to a host cell transformed with an expression vector according to invention. Host cells may be prokaryotic or eukaryotic, including but not limited to bacteria, yeasts, insect cells, mammalian cells, including cell lines, which are commercially available.

The invention is also directed to a process for manufacturing a purified polypeptide comprising:

a) transfection of a host cell with an expression vector to obtain expression of the polypeptide, and

b) isolation and purification of the polypeptide from the transfected host cell.

Purification of the polypeptide may be accomplished by standard methods for purification of a membrane or soluble proteins. The invention also relates to a pharmaceutical composition comprising as an active agent at least one molecule according to the invention. The pharmaceutical compositions of the invention are useful for treating immune-related pathologies and, in particular, they are useful for modulating immune responses. In a preferred embodiment, the pharmaceutical compositions are useful to enhance development of CD4 or CD8 T-cell populations. In another preferred embodiment, the pharmaceutical compositions of the invention are also useful to suppress the development of CD4 or CD8 T-cell populations.

The pharmaceutical composition of the invention comprise as an active agent a LAP agonist. In another preferred embodiment, the pharmaceutical composition of the invention comprises as an active agent a LAP antagonist. A LAP agonist is any molecule that mimics the effect of LAP binding when it binds to the target EP motifs and a LAP antagonist is any molecule that inhibits the affect of LAP binding when it binds to the target EP motif.

The invention also includes the use of a molecule according to invention to manufacture a pharmaceutical composition useful for treating immune-related pathologies or for modulating immune responses. The invention relates to the manufacture of a pharmaceutical composition enhancing the development of CD4 or CD8 T-cell populations. The invention further relates to the manufacture of a pharmaceutical composition for suppressing development of CD4 or CD8 T-cell populations.

In a preferred embodiment, the molecule is a LAP agonist. In a preferred embodiment, the molecule is a LAP antagonist.

The invention also includes a method for screening drugs comprising the steps of:

contacting the drug candidate with a molecule according to the invention in the presence of a target EP motif, and

measuring the resulting binding of the molecule to the target.

The method for screening drugs allows the screening of drugs selected from the group comprising drugs able to activate T-cell, drugs enhancing the development of CD4 or CD8 T-cell populations, drugs suppressing development of CD4 or CD8 T-cell populations, and drugs active in platelet activation. Preferably, the molecule is a LAP polypeptide.

The invention also relates to antibodies directed to a specific epitope of the polypeptide identified by SEQ ID NO:1. In preferred embodiments, the antibodies are monoclonal antibodies or polyclonal antibodies or Fab, Fab′, F(ab′) or Fv fragments thereof.

The invention also comprises a monoclonal or polyclonal antibody or monoclonal or polyclonal antibody fragments or derivatives that specifically binds a peptide of SEQ ID NO:1, the monoclonal or polyclonal antibody derivative being selected from the group consisting of a monoclonal or polyclonal antibody conjugated to a cytotoxic agent or a radioisotope, and Fab, Fab′ or F(ab′)₂ fragments of the monoclonal or polyclonal antibody conjugated to a cytotoxic agent or radioisotope.

Antibody fragments are regions from the polyclonal or monoclonal antibodies sequences recognising at least one epitope present in the peptide of SEQ ID NO:1, which maintain the binding capacity for at least one of the epitopes. Antibody derivatives are entire or fragment antibodies labelled with chemical or biological entities to be easily detected. Chemical or biological entities may be enzymes, fluorescent labels, coloured particles and the like.

The invention relates also to a hybridoma cell line producing a monoclonal antibody according to the invention. The invention is also directed to a therapeutic composition comprising as active ingredient an antibody according to the invention. The invention also relates to use of the antibodies in a method for purifying, identifying or quantifying a polypeptide or its homologs.

The invention relates to use of the antibodies to screen compounds active in intracellular signaling mediated by cell surface receptor. The invention also relates to use of the antibodies to screen compounds active in T-cell activation or regulation of the expansion of activated T-cells. The invention is also directed to use of the antibodies to screen compounds active in platelet activation.

The present invention also relates to use of the antibodies for manufacturing a therapeutic composition useful for treating immune-related pathologies. The invention also relates to use of the antibodies for manufacturing an immunomodulatory pharmaceutical composition.

### EXAMPLES

1.1 LAG-3 and MHC Class II are Expressed in GSL Complexes on the Surface of Human Activated T cells

GSL complexes (raft microdomains) were isolated in a low-density fraction at the interface between the 35% and 5% fractions of a discontinuous sucrose gradient, as described by Montixi et al. (Montixi, C., Langlet, C., Bernard, A. M., Thimonier, J., Dubois, C., Wurbel, M. A., Chauvin, J. P., Pierres, M. and He, R. T., Engagement of T cell receptor triggers its recruitment to low-density detergent-insoluble membrane domains The EMBO Journal 1998. 17:5334-5348). Twelve fractions of the gradient were analyzed by Western-blotting. LAG-3, DR-α as well as p561ck were detected in fraction 9, representing the GSL complex isolates, and were not detected following addition of 0.2% saponin (cholesterol depletion leading to raft disruption) to 1% Triton X-100. CD45, a phosphotyrosine phosphatase known to be excluded from raft microdomains, was used as a negative control. Thus, LAG-3 is present in raft microdomains before engagement of the TCR by specific mAb or peptide/MHC complexes.

In addition, MHC class II (DR-α) molecules were present in raft microdomaine on activated T cells. Partitioning of MHC class II into the raft fraction has been reported to occur in the myelomonocytic THP-1 cells following their crosslinking with antibodies and to be mandatory for protein tyrosine kinase (PTK) activation (Huby, R. D. J., Dearman, R. J. and Kimber, I., Intracellular phosphotyrosine induction by major histocompatibility complex class II requires co-aggregation with membrane rafts J. Biol. Chem. 1999. 274: 22591-22596). In B cells, MHC class II were found to be constitutively present in rafts and this concentration of MHC class II molecules facilitates antigen presentation (Anderson, H. A., Hiltbold, E. M. and Roche, P. A., Concentration of MHC class II molecules in lipid rafts facilitates antigen presentation Nature Immunol. 2000. 1: 156-162).

The presence of LAG-3 in raft microdomains before engagement of the TCR suggests a close association with CD3/TCR complexes and explains, in part, previous observations where LAG-3 was found to be co-clustered with CD3/TCR complexes and also with CD8 in co-capping experiments (Hannier, S. and Triebel, F., The MHC class II ligand LAG-3 is co-distributed with CD8 and CD3/TCR molecules after their engagement by mAbs or peptide/MHC class I complexes Int. Immunol. 1999. 11: 1745-1752). 1.2 Isolation of a Novel Human Protein, LAP, Interacting with LAG-3

An interaction screening was performed by using the yeast two-hybrid system to identify proteins that bind to the intracellular domain of human LAG-3 in vivo. First, it was verified that no LAG-3 construct in pLex or pLex/NLS displayed any lacZ reporter gene activity in yeast cells expressing pGAD without insert. This indicated that LAG-3 does not show any non-specific binding to DNA sequences leading to GAL promoter activation. Then, strain L40 was transformed with pLex/NLS-hLAG-3/I to screen about 2×10⁵ colonies of the human activated T-cell cDNA library. Around 200 colonies that grew on histidine-free drop-out medium were selected, replaced onto selective medium and assayed for β-galactosidase expression. From these, 13 showed reporter gene activities.

To confirm the specificity of these interactions, the plasmid DNA from selected clones was isolated and used for transformation of the strain AMR70, which were then mated with strain L40 containing either the bait plasmid pLex/NLS-hLAG-3/I or a control plasmid (pLax-Lamin or pLex/NLS-RalB). Three specific clones were obtained showing strong interaction with hLAG-3/I (signals appeared in less than 2 hrs) and not with Lamin or RalB.

The inserts of these clones were submitted to restriction mapping and sequence analysis. The three cDNAs were found to encode a unique partial (i.e., lacking the ATG translation initiation codon) sequence of 243 amino acids, termed LAP (not shown). This novel molecule has some homology with the C terminal region of the TCP-10 protein previously cloned in human (Islam, S. D., Pilder, S. H., Decker, C. L., Cebra-Thomas, J. A. and Silver, L. M., The human homolog of a candidate mouse t complex responder gene: conserved motifs and evolution with punctuated equilibria, Human Molecular Genetics 1993. 2: 2075-2079 and Bibbins, K. B., Tsai, J. Y., Schimenti, J., Sarvetnick, N., Zoghbi, H. Y., Goodfellow, P. and Silver, L. M., Human homologs of two testes-expressed loci on mouse chromosome 17 map to opposite arms of chromosome 6, Genomics 1989. 5: 139-143) and mouse (Schimenti, J., Cebra-Thomas, J. A., Decker, C. L., Islam, S. D., Pilder, S. H. and Silver, L. M., A candidate gene family for the mouse t complex responder (Tcr) locus responsible for haploid effects on sperm function, Cell 1988. 55: 71-78; Ewulonu, U. K., Snyder, L., Silver, L. M. and Schimenti, J. C., Promoter mapping of the mouse Tcp-10bt gene in transgenic mice identifies essential male germ cell regulatory sequences, Molecular Reproduction and Development 1996. 43: 290-297 and Cebra-Thomas, J. A., Decker, C. L., Snyder, L. C., Pilder, S. H. and Silver, L. M., Allele- and haploid-specific product generated by alternative splicing from a mouse t complex responder locus candidate, Nature 1991. 349: 239-241) TCP-10 is a T-complex responder (TCP) gene that may play a role in the transmission ratio distortion phenotype. A region of LAP is 56% identical to the 181 C-terminal residues of human TCP-10 protein and 66% identical to the 106 C-terminal residues of the murine TCP-10 protein.

The 5′ end of the LAP cDNA was further extended by 5′RACE cloning starting from PHA-blasts mRNA. Analysis of the LAP cDNA revealed a nucleotide sequence of 1353 bases that contains a single open reading frame (ORF) of 372 amino acids. This ORF starts at position 70 and ends with the translation stop codon, TGA, located at nt 1186.

It was found that this LAP sequence is 99% identical (9 nt mismatches including 4 in the coding region with a single a.a. difference at the carboxy-terminus) to the 3′ end of the recently published CPAP (centrosomal P4.1-associated protein) molecule, which is part of the γ-tubulin complex (Hung, L. Y., Tang, C. C. and Tang, T. K., Protein 4.1 R-135 interacts with a novel centrosomal protein (CPAP) which is associated with the gamma-tubulin complex, Mol. Cell. Biol. 2000. 20: 7813-7825). These 9 nt mismatches were also found on several EST sequences, confirming the differences observed between CPAP and LAP. (Hung, L. Y., et al. Mol. Cell. Biol. 2000.20: 7813-7825).

Sequence identity of TCP-10, CPAP and LAP proteins is restricted to two conserved regions at the COOH-terminus. One carries a leucine zipper, which may form a series of heptads. Repeats involved in coiled-coil formations, and the second contains unusual glycine repeats (Hung, L. Y., et al. Mol. Cell. Biol. 2000. 20: 7813-7825).

Additional tests were performed to verify whether human LAP could bind to the murine LAG-3 IC region. A weak interaction was observed between these two heterologous proteins with a small activation of the HIS3 gene, but no detectable LacZ activity. Next, it was examined which region of human LAG-3 interacts with LAP. The binding of LAP with HLAG-3/IΔC and hLAG-3/EP constructs was tested in yeast cells and it was found that LAP indeed binds specifically with the short C-terminal region of LAG-3 containing the EP-rich region. These results illustrating interaction of LAP and LAG-3 proteins (a) are shown in Table 1.

TABLE 1

| fused to LexA BD | | | fused to Gal4 AD | | |
|---|---|---|---|---|---|
| | LAG-3 regions | — | Lamin | RalB | LAP |
| hLAG-3/I | R457 to L503 | — | − | − | ++++ |
| NLS-hLAG-3/I | R457 to L503 | — | − | − | +++++ |
| NLS-mLAG-3/I | L456 to L507 | — | − | − | + |
| NLS-hLAG-3/I?C | R457 to E481 | — | − | − | +/− |
| hLAG-3/EP | E478 to L503 | — | − | − | ++ |
| NLS/hLAG-3/EP | E478 to L503 | — | − | − | ++++ |

Where LAG proteins are hLAG-3 and mLAG-3, IC regions were expressed as fusion proteins to the LexA DNA binding domain (LexA BD) in the pLex vector containing or not a nuclear localization sequence (NLS). The pGAD vector encoded the GAL4 activation domain (GAL4 AD) alone or fused to LAP or an unrelated protein (Lamin or RaIB). Two procedures for interaction studies were performed: (i) co-transfection of yeast strain L40 with the two indicated plasmid combinations shown, (ii) transformation of strain L40 with a pLex construct which are then mated with strain AMR70 transformed with a pGAD construct.

A demonstration of in vitro binding between LAP and hLAG-3 proteins, LAP linked to GST or GST alone were expressed in bacteria and bound to glutathione-Sepharose beads was performed as described hereafter.

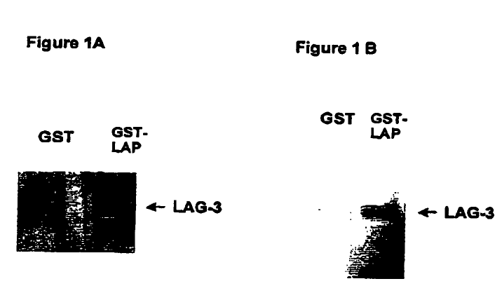

Bound proteins were incubated with total cell lysates prepared from PHA-activated T lymphocytes. The results demonstrate that the LAG-3 protein was specifically precipitated from the T-cell lysate when using affinity beads containing the LAP protein (FIG. 1A). The control GST beads did not precipitate any detectable LAG-3 protein from the T-cell lysate. Therefore, LAG-3 binds specifically to the LAP protein in vitro, in agreement with the data obtained from the yeast two-hybrid screening procedure.

A direct binding assay in which the in vitro-translated LAG-3 protein was tested for interaction with beads bound to GST-LAP or GST alone was performed to verify that the interaction between LAP and LAG-3 proteins in both the yeast two-hybrid system and in T-cell lysates does not require an additional adaptor protein.

Affinity beads containing the GST-LAP fusion protein pulled down the LAG-3 protein in a specific manner as shown in FIG. 1B. This supports the existence of a specific direct physical interaction between LAP and LAG-3 proteins without the need for the presence of a third adaptor protein.

Overall, the interaction between LAP and hLAG-3 has been confirmed both in vivo and in vitro using recombinant LAP protein. In particular, the LAP protein was able to bind LAG-3 in lysates of activated T cells. This interaction was specific and also observed vice versa using in vitro translated recombinant LAG-3.

1.3 The C-terminus Region of LAP Binds the EP Region of hLAG-3

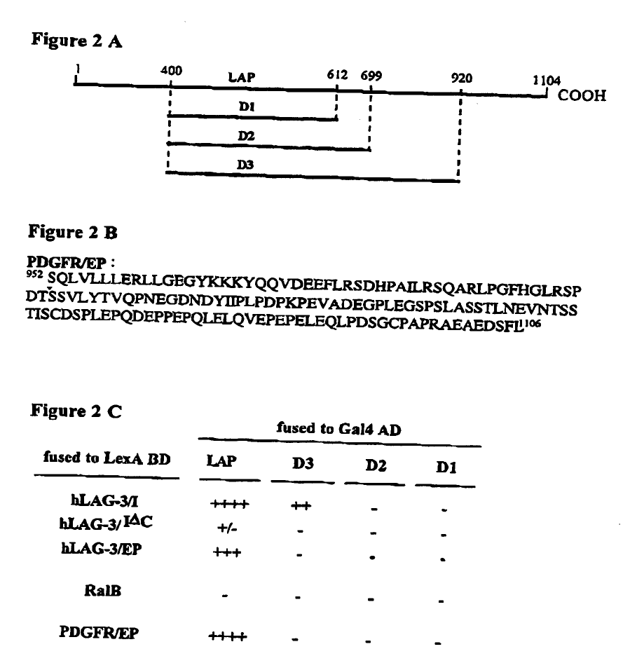

Deletion mutants of the LAP cDNA were constructed to determine the region of the LAP protein that contains the LAG-3 binding site (FIG. 2A). The binding of these mutants with hLAG-3/I, hLAG-3/IΔC and hLAG-3/EP were tested with Ral B as a negative control. Deletion of the extreme C-terminal regions (mutant D3) already abolished some binding activity (FIG. 2C), while the shorter constructs (D1 & D2) did not bind to hLAG-3 at all.

Thus, the binding site for LAP on the EP motifs is located in its C-terminal region. LAP functions to cluster rafts into the immunological synapse following TCR engagement, a phenomenon that requires the polarization of actin and microtubules (Simons, K. and Toomre, D., Lipid rafts and signal transduction Nature 2000. 1: 31-39).

1.4 LAP Binds to the Intracytoplasmic Region of the PDGF Receptor Containing an EP Motif

The PDGF receptor (Claesson-welsh, L., A. Eriksson, A.Morén, L. Severinsson, B. Ek, A. Ostman, C. Betsholtz and C. H. Heldin, cDNA cloning and expression of a human platelet-derived growth factor (PDGF) receptor specific for B-chain-containing PDGF molecules, Mol. Cell. Biol. 1988. 8: 3476-3486) has a long intracytoplasmic tail containing numerous motifs known to be involved in signalling. A repeated EP motif not known to be involved in transduction signalling was found in its C-terminal region (FIG. 2B).

Surprisingly, the LAP protein could bind to this EP motif-containing segment. Thus, LAP interactions with other membrane receptor intracytoplasmic regions containing the EP motif have crearly been identified, since this work shows that it binds to the PDGFR intracellular region in addition to hLAG-3 and mLAG-3. Thus, this EP motif appears as a common transduction motif, that can be used by other functionally important receptors.

1.5 LAP is a 45 kDa Protein Expressed in all Tested Human Cells

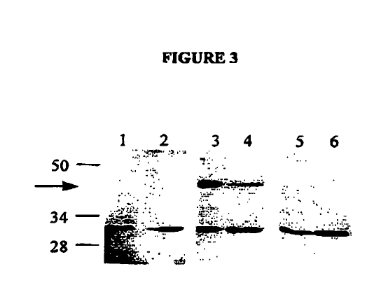

Total cell lysates were analyzed by Western blotting with a rabbit polyclonal serum rose against a LAP peptide with no sequence homology with TCP-10 to determine the size and expression of the LAP protein. Two bands at 30- and 45 kDa were detected in PBMCs on activated T-cells (FIG. 3). The 30 kDa band was shown to be non-specific since it was also detected using the preimmune serum (FIG. 3). The 45 kDa band corresponds to LAP as it was no longer detected following pre-incubation of the immune serum containing the LAP peptide (10-6 at 4° C. for 1 hr) (FIG. 3) while pre-incubation with a control peptide had no effect (data not shown). In addition, this 45 kDa band was found in cytoplasmic but not in nucleic T cell extracts.

These results clearly indicate that LAP is expressed as a 45 kDa cytoplasmic protein in PBMCs and in activated T cells with a higher expression level in the latter cells.

Western blotting was also performed with total cell lysates of the Jurkat T cell line, two EBV-transformed B cell lines and a renal cell carcinoma cell line (RCC7).

LAP is also expressed in these cell lines as a 45 kDa protein with lower expression in PBMC. LAP is thus expressed in T and non-T hematopoietic cell lines as well as in non-hematopoietic cell lines. In addition, LAP was detected in different untransformed human tissues, including the lung, liver, kidney, testes (no overexpression, in contrast to CPAP), pancreas and heart, but not in the spleen and brain (data not shown).

1.6 Two RNA Species are Derived from the LAP Gene

The LAP gene was first analyzed by digesting DNA from different cell lines and PBLs, Southern blotting and hybridizing using the LAP cDNA as a probe. Unique EcoRI (5.5 kb), Hind III (9 kb) and Xho I (>12 kb) fragments were found indicating that the LAP or CPAP gene is either present in the human genome as a single copy gene or represents two closely related genes (data not shown).

Total and poly-A⁺ RNA samples of PHA-blasts were run on a denaturing agarose gel and analyzed by Northern blotting. The LAP RNA seemed to be rarely expressed, as it was only detected by using 15 μg of poly-A⁺ RNA while not being detected in total RNA samples (up to 20 μg, data not shown). Two faint bands hybridized with the labelled cDNA LAP probe, one with a size of 4.5 kb and a weaker one at 1.8 kb. As these two bands correspond exactly to the sizes of the 28S and 18S rRNA, the blot was then rehybridized with saturating amounts of ribosomal RNA (10 μg/ml) added to prevent non-specific binding of the probe to the remaining rRNA in the sample. The same result was obtained.

Since these two signals were only seen with highly purified poly-A⁺ RNA and not with total RNA samples containing a greater amount of rRNA, I concluded that LAP was specifically expressed as a 1.8 kb mRNA. The stronger 4.5 kb signal may correspond to CPAP, which has been shown to be weakly expressed in most tissues, except testis (Hung, L. Y., Tang, C. C. and Tang, T. K., Protein 4.1 R-135 interacts with a novel centtosomal protein (CPAP) which is associated with the gamma-tubulin complex, Mol. Cell. Biol. 2000. 20: 7813-7825).

Thus, LAP is a new human protein expressed in all tested human cells and derived from a rare mRNA. It appears that LAP and CPAP are derived from either a single gene or two closely related genes strongly expressed in the testes for the CPAP mRNA (4.5 kb) and weakly expressed in other cells as two messages (4.5 kb and 1.8 kb) coding for CPAP and LAP, respectively.

The specific immunoprecipitation of LAG-3 by LAP-GST beads from activated T lymphocyte lysates indicates that the two overlapping 150 kDa CPAP (Hung, L. Y., Tang, C. C. and Tang, T. K., Protein 4.1 R-135 interacts with a novel centrosomal protein (CPAP) which is associated with the gamma-tubulin complex, Mol. Cell. Biol. 2000. 20: 7813-7825) and 45 kDa LAP proteins have different functions, that is binding to Γ-tubulin in the centrosome especially in testis cells (CPAP) or binding to EP motifs present on membrane expressed receptors (LAP).

EP motifs are rare in human proteins, and the specific binding of LAP on such motifs has important biological significance for signal transduction and/or coupling of clustered rafts to the microtubule networks.

2.1 Plasmid Construction

The hLAG-3/I and mLAG-3/I fragments encode the full length intracellular region of human LAG-3 and murine LAG-3, respectively. The hLAG-3/IΔC encodes the intracellular domain of human LAG-3 deleted of its 22 C-terminal amino acids (ΔC) whereas hLAG-3/EP codes only for the EP-rich region located at the end of the C-terminal part of hLAG-3. The PCR products were cloned into the two hybrid-vectors pBMT116 (pLex) or a derivative containing an additional Nuclear Localization Sequence (pLex/NLS) (Vojtek, A. B. and Hollenberg, S. M., Ras-Raf interaction: two-hybrid analysis, Methods Enzymol. 1995. 255: 331-342) in frame with the LexA DNA binding protein yielding the following constructs:

pLex-hLAG-3/I and pLex/NLS-hLAG-3/I (from R⁴⁵⁷ to L⁵⁰³)

pLex/NLS-mLAG-3 (from L⁴⁵⁶ to L⁵⁰⁷)

pLex-hLAG-3/IΔC and pLex/NLS-hLAG-3/IΔC (from R⁴⁵⁷ to E⁴⁸¹)

pLex-hLAG-3/EP and pLex/NLS-hLAG-3/EP (from E⁴⁷⁸ to L⁵⁰³)

2.2 Two-Hybrid Screen and Interaction Analysis

Yeast, medium and two-hybrid procedures were handled according to published methods (Vojtek, A. B. and Hollenberg, S. M., Ras-Raf interaction: two-hybrid analysis, Methods Enzymol. 1995. 265: 331-342; Kaiser, C., Michaelis, S. and Mitchell, A., Methods in yeast genetics Cold Spring Harbor Laboratory 1994). For the two hybrid-screen, a human activated PBL library cloned in the pGAD-1318 vector (Hybrigenics, Paris, France) which contains the activation domain of GAL4 under the control of the entire ADH1 strong yeast promoter was used. For library screening, yeast strain L40 which contains the LacZ and HIS3 reporter genes downstream of the binding sequence of LexA was sequentially transformed with pLex/NLS-hLAG-3/I and 60 μg of the human activated T cell library using the lithium acetate method. Double transformants were plated on yeast drop-out medium lacking tryptophan, leucine and histidine, and incubated at 30° C. for 3 days. Positive colonies His⁻ were patched on selective plates for growth and then replicated on Whatman 40 paper. The β-galactosidase activity was tested by a filter assay.

For interaction studies, two methods were used: by co-transformation of strain L40 with pairs of pLex and pGAD vectors, or by mating the strain L40 expressing a pLex vector with the strain AMR70 containing a pGAD vector. In both cases, binding was tested for growth in histidine-deficient medium and for β-galactosidase activity. Signals described as being negative were not detected even after 3 days or 24 hrs for the HIS3 and LacZ reporter genes, respectively. No discrepancy was observed between the histidine auxotrophy and the β-galactosidase tests.

2.3 Protein Expression and Purification

LAP polypeptide was expressed as a glutathione S-transferase (GST) fusion protein in Escherichia coli and immobilized on affinity matrix beads. Briefly, fresh overnight cultures of E. Coli HB101 or XL-1 blue cells harboring the pGEX plasmid expressing GST or GST-LAP proteins were diluted 1:10 in Luria-Bertani (LB) broth supplemented with 20 μg/ml ampicillin and the cultures were grown for 3 h with 0.1 mM IPTG (Sigma, St. Louis, Mo.). Cell pellets were collected by centrifugation and lysed in Tris buffer containing 1% NP-40 and anti-proteases. The soluble fraction was prepared by centrifugation at 10,000 g for 15 min at 4° C. The GST and recombinant GST fusion proteins were purified by coupling to Glutathione Sepharose 4B beads (Pharmacia, Uppsala, Sweden) by gentle mixing at 4° C. for 40 min followed by extensive washing. The protein-bound affinity beads were analyzed and quantitated by Coomassie blue R-250 staining following SDS-PAGE analysis.

2.4 Preparation of Cell Lysates and in Vitro Binding Assays

Human PBMCs were isolated from venous blood by Ficoll-Paque density gradient centrifugation. T lymphocytes were obtained by stimulating PBMCs with 1 μg/ml of PHA-P (Wellcome, Beckenham, UK) at 37° C. and 10% CO₂ in complete culture medium (RPMI 1640 supplemented with 10% heat inactivated human AB serum, 4 mM L-glutamine, 1 mM pyruvate, 0.2 mM NaOH, 50,000 IU penicillin and 50 mg/ml streptomycin). Whole cell lysates were prepared in Tris cell lysis buffer containing 1% NP-40 and anti-proteases after 3 days of culturing.

The hLAG-3 protein was synthesized in vitro using the T7-coupled rabbit reticulocyte lysate system (TNT, Promega, Madison, Wis.). Equal amounts of GST-LAP or control GST proteins immobilized on beads were incubated for 3 hrs at 4° C. with direct whole cell lysates (after centrifugation of nuclei) or with the in vitro translated hLAG-3 protein in a binding buffer (20 mM Tris-HCl pH 7.5, 50 mM NaCl, 1 mM PMSF, 1 μg/ml leupeptin, 1 μg/ml aprotinin). Bound proteins were then extensively washed in PBS buffer and analyzed by Western blotting.

2.5 Cell Lines and Antibodies

The Jurkat T cell line and the Epstein Barr Virus (EBV)-transformed B cell line were grown in complete 1640 RPMI culture medium at 37° C. and 6% CO₂. RCC7 (a renal cell carcinoma cell line, (Gaudin, C., Kremer, P., Angevin, E., Scott, V. and Triebel, F., A HSP70-2 mutation recognized by cytolytics T lymphocytes on a human renal cell carcinoma, J. Immunol. 1999.162: 1730-1738) were cultivated in complete DMEM medium at 37° C. and 6& CO₂.

A polyclonal serum was raised against a peptide (SPREPLEPLNFPDPEYK) derived from the deduced amino-acid sequence of LAP by immunizing rabbits with three injections of peptide-BSA (Neosystem, Strasbourg, France).

2.6 Western Blot

10⁶ cells were washed and lysed at 4° C. for 60 min in 100 μl Tris cell lysis buffer. Cell debris were removed by 10 min centrifugation at 10,000 g and the lysates heat-denaturated in SDS sample buffer for 5 min. Total cell lysates were separated by SDS-PAGE and transferred to nitrocellulose membranes. Membranes were saturated with 5% dry milk for 1 hr at 37° C. and incubated with primary antibody diluted 1:3000 in TBS for 1.5 hr with slow agitation. After incubating the membranes with the GAR-peroxidase secondary antibody, the signal was detected by enhanced chemiluminescence (ECL, Amersham, Buckinghamshire, UK). A commercial Western blot containing 75 μg of total cellular protein from eight different human tissues (Chemicon, Temecula, USA) was used to determine the tissue distribution of LAP.

In describing amino acid sequences, the term “homolog” means that the amino acid sequence is at least 50% homologous or any integer percentage thereof, preferably at least 80% homologous and most preferably 90% homologous. With regard to a “fragment” or a “derivative thereof”, this means the sharing of at least 50% sequence identity or any integer percentage thereof as determined by the Clustal method of alignment.

1) A molecule binding to a target comprising an EP motif having the following sequence:
(X-(EP)ₙ-Y-(EP)ₘ-Z)ₚ
wherein X, Y and Z may be identical or different and comprise a sequence of 0 to 10 amino acids, identical or different, n and m are integers between 0 to 20, preferably between 3 to 10, with at least one of n or m being different from 0, and p is an integer between 1 and 10.

2) The molecule according to claim 1, which binds to an EP motif selected from the group consisting of EPEPEPEPEPEPEPEPEP (SEQ ID N^{o} 3), EPEPEPQLEPEP (SEQ ID N^{o} 4), EPQDEPPEPQLELQVEPEPELEQ (SEQ ID N^{o} 5), and EPEPEPEPEPEP (SEQ ID N^{o} 6).

3) The molecule according to claim 1, which binds to an amino acid sequence comprising at least 5 EP motifs over a 19 amino acid segment.

4) The molecule according to claim 1, wherein the molecule is selected from the group consisting of a peptide, a polypetide or a protein.

5) The polypeptide according to claim 4, comprising the amino acid sequence of LAP identified by SEQ ID No.:1, a homolog, a fragment or a derivative thereof.

6) The polypeptide according to claim 4, comprising the carboxy-terminal amino acid sequence of LAP identified by SEQ ID No.:2, a homolog, a fragment or a derivative thereof.

7) A nucleic acid molecule comprising a polynucleotide sequence coding a polypeptide according to claim 4.

8) The nucleic acid molecule according to claim 7, comprising the polynucleotide sequence identified by SEQ ID No.:8, a fragment or a derivative thereof.

9) An expression vector comprising a nucleic acid molecule according to claim 7.

10) An expression vector comprising a nucleic acid molecule according to claim 8.

11) A host cell transformed with an expression vector according to claim 9.

12) A host cell transformed with an expression vector according to claim 10.

13) A process for manufacturing a molecule binding to a target comprising an EP motif having the following sequence:
(X-(EP)ₙ-Y-(EP)ₘ-Z)ₚ
wherein X, Y and Z may be identical or different and comprise a sequence of 0 to 10 amino acids, identical or different, n and m are integers between 0 to 20, preferably between 3 to 10, with at least one of n or m being different from 0, and p is an integer between 1 and 10, comprising:
a) transfection of a host cell with an expression vector according to claim 5 to obtain expression of the polypeptide, and
b) isolation and purification of the polypeptide from the transfected host cell.

14) A pharmaceutical composition comprising as active agent at least one molecule according to claim 1.

15) The pharmaceutical composition according to claim 14, wherein said molecule is a LAP agonist.

16) The pharmaceutical composition according to claim 14, wherein said molecule is a LAP antagonist.

17) A method of treating immune-related pathologies comprising administering a therapeutically effective amount of a molecule according to claim 1 to a patient in need thereof.

18) A method of treating the immune response comprising administering a therapeutically effective amount of a molecule according to claim 1 to a patient in need thereof.

19) A method of enhancing the development of CD4 or CD8 T-cell populations comprising administering a therapeutically effective amount of a molecule according to claim 1 to a patient in need thereof.

20) A method of suppressing the development of CD4 or CD8 T-cell populations comprising administering a therapeutically effective amount of a molecule according to claim 1 to a patient in need thereof.

21) The method according to claim 7, wherein said molecule is a LAP agonist.

22) The method according to claim 7, wherein said molecule is a LAP antagonist

23) A method for screening drugs comprising:
contacting a candidate drug with a molecule according to claim 1 in the presence of a target EP motif, and measuring resulting binding of the molecule to the target.

24) The method according to claim 23, wherein the drugs are selected from the group consisting of drugs able to activate T-cell, drugs enhancing development of CD4 or CD8 T-cell populations, drugs suppressing development of CD4 or CD8 T-cell populations and drugs active in platelet activation.

25) The method according to claim 23, wherein the molecule is a LAP polypeptide.

26) Antibodies directed to a specific epitope of a polypeptide selected from the group consisting of polypeptides or peptides identified by SEQ ID NO:1, SEQ ID NO:2, SEQ ID NO:3, SEQ ID NO:4, SEQ ID NO:5, SEQ ID NO:6, and SEQ ID NO:9.

27) The antibodies according to claim 26, wherein the antibodies are monoclonal antibodies or Fab, Fab′, F(ab′) or Fv fragments thereof.

28) A monoclonal antibody or a monoclonal antibody derivative that specifically binds a peptide selected from the group consisting of polypeptides or peptides identified by SEQ ID NO:1, SEQ ID NO:2, SEQ ID NO:3, SEQ ID NO:4, SEQ ID NO:5, SEQ ID NO:6 and SEQ ID NO:9, the monoclonal antibody derivative being selected from the group consisting of a monoclonal antibody conjugated to a cytotoxic agent or a radioisotope, and Fab, Fab′ or F(ab′)₂ fragments of said monoclonal antibody conjugated to a cytotoxic agent or radioisotope.

29) A hybridoma cell line producing the monoclonal antibody of claim 28.

30) A therapeutic composition comprising as an active ingredient an antibody according to claim 26.


SEQUENCE LISTING (10 sequences)

SEQ ID NO: 1 - protein, 372 aa, Homo sapiens [sequence omitted]

SEQ ID NO: 2 - protein, 135 aa, Homo sapiens
  PEPTIDE (1)..(135): COOH-terminal peptide from LAP protein
QRVIYYYAAAQTTHTTYPEGLEVLHFSSGQIEKHYPDGRKEITFPDQTVKNLFPDGQEESIFPDGTIVRVQRDGNKLIEFNNGQRELHTAQFKRREYPDGTVKTVYANGHQETKYRSGRIRVKDKEGNVLMDTEL

SEQ ID NO: 3 - protein, 18 aa, Artificial Sequence
  Description of Artificial Sequence Synthetic amino acid motif sequence
EPEPEPEPEPEPEPEPEP

SEQ ID NO: 4 - protein, 12 aa, Artificial Sequence
  Description of Artificial Sequence Synthetic amino acid motif sequence
EPEPEPQLEPEP

SEQ ID NO: 5 - protein, 23 aa, Artificial Sequence
  Description of Artificial Sequence Synthetic amino acid motif sequence
EPQDEPPEPQLELQVEPEPELEQ

SEQ ID NO: 6 - protein, 12 aa, Artificial Sequence
  Description of Artificial Sequence Synthetic amino acid motif sequence
EPEPEPEPEPEP

SEQ ID NO: 7 - protein, 486 aa, Homo sapiens [sequence omitted]

SEQ ID NO: 8 - DNA, 1353 bp, Homo sapiens
  misc_feature (1)..(1353): LAP cDNA Open reading frame
gaaattgcagacttcgaacaacagaaagcaaaagaattagctcgaatagaagagtttaaaaaggaggagatgaggaagctacaaaaggaacgtaaagtttttgaaaagtatactacagctgcaagaacttttccagataaaaaggaacgtgaagaaatacagactttaaaacagcaaatagcagatttacgggaagatttgaaaagaaaggaaaccaaatggtcaagtacacacagccgtctcagaagccagatacaaatgttagtcagagagaacacagacctccgggaagaaataaaagtgatggaaagattccgactggatgcctggaagagagcagaagccatagagagcagcctcgaggtggagaagaaggacaagcttgcgaacacatctgttcgatttcaaaacagtcagatttcttcaggaacccaggtagaaaaatacaagaaaaattatcttccaatgcaaggcaatccacctcgaagatccaagtctgcacctcctcgtgatttaggcaatttggataagggacaagctgcctctcccagggagccacttgaaccactgaacttcccagatcctgaatataaagaggaggaggaagaccaagacatacagggagaaatcagtcatcctgatggaaaggtggaaaaggtttataagaatgggtgccgtgttatactgtttcccaatggaactcgaaaggaagtgagtgcagatgggaagaccatcactgtcactttctttaatggtgacgtgaagcaggtcatgccagaccaaagagtgatctactactatgcagctgcccagaccactcacacgacatacccggagggactggaagtcttacatttctcaagtggacaaatagaaaaacattacccagatggaagaaaagaaatcacgtttcctgaccagactgttaaaaacttatttcctgatggacaagaagaaagcattttcccagatggtacaattgtcagagtacaacgtgatggcaacaaactcatagagtttaataatggccaaagagaactacatactgcccagttcaagagacgggaatacccagatggcactgttaaaaccgtatatgcaaacggtcatcaagaaacgaagtacagatccggtcggataagagttaaggacaaggagggtaatgtgctaatggacacggagctgtgacgatcctcatgtgatcatgaagtaacagtaactgactttttatgttaaaaaatgtacatttactgtggattctgtttaatttattgtgtatgtgtggggaaaagattggattctaaaataaaagtttaccctgtggcatcttcatttttatattctttgaaatgc

SEQ ID NO: 9 - protein, 17 aa, Artificial Sequence
  Description of Artificial Sequence Synthetic peptide
SPREPLEPLNFPDPEYK

SEQ ID NO: 10 - protein, 152 aa, Artificial Sequence
  Description of Artificial Sequence Synthetic fusion protein
SQLVLLLERLLGEGYKKKYQQVDEEFLRSDHPAILRSQARLPGFHGLRSPDTSSVLYTVQPNEGDNDYIIPLPDPKPEVADEGPLEGSPSLASSTLNEVNTSSTISCDSPLEPQDEPPEPQLELQVEPEPELEQLPDSGCPAPRAEAEDSFL